(19) United States
(12) Patent Application Publication
(10) Pub. No.: US 2018/0092544 A1
(43) Pub. Date: Apr. 5, 2018

(54) SYSTEM AND METHOD FOR ANGIOGRAPHY

(71) Applicants: Hsiang-Chen CHUI, Tainan City (TW); METAL INDUSTRIES RESEARCH & DEVELOPMENT CENTRE, Kaohsiung (TW)

(72) Inventors: Ming-Hui CHEN, Kaohsiung City (TW); Hsiang-Chen CHUI, Taichung City (TW); Wei-Te CHEN, Changhua County (TW)

(21) Appl. No.: 15/369,901

(22) Filed: Dec. 6, 2016

(30) Foreign Application Priority Data
Oct. 5, 2016 (TW) 105132218

(51) Int. Cl. A61B 5/00; A61B 1/07; A61B 8/08

(52) U.S. Cl. CPC A61B 5/0095; A61B 1/07; A61B 2562/028; A61B 8/0891; A61B 8/5207; A61B 5/6851

(57) ABSTRACT

The system comprises an optical fiber, a light emitting module, an ultrasound detector and an imaging system. The optical fiber has a first end and a second end. The second end of the optical fiber is configured to enter a blood vessel of a human body. The light emitting module emits a laser ray into the first end of the optical fiber, and the laser ray diffuses into a human tissue through the optical fiber to generate an ultrasound signal. The ultrasound detector is disposed corresponding to the second end of the optical fiber for receiving the ultrasound signal. The imaging system is coupled to the ultrasound detector and generates an image with respect to the blood vessel according to the ultrasound signal.

### RELATED APPLICATIONS

This application claims priority to Taiwan Application Serial Number 105132218 filed Oct. 5, 2016, which is herein incorporated by reference.

### BACKGROUND

### Field of Invention

The present invention relates to an angiography method. More particularly, the present invention relates to an intrusive angiography method based on photoacoustic effect.

### Description of Related Art

The World Health Organization (WHO) predicts that more than twenty-three million people will die due to cardiovascular diseases annually in the world before year 2030. Ministry of health and welfare in Taiwan predicts that number of the patient suffered from cardiovascular diseases will increase 0.5 million per year. The number of patients having cardiovascular disease has been the highest in the world in an aspect of demand side of medical market. The product demand for all kinds of wire/catheter has been increased for years, and the scale of the global market is nearly 28.6 billion dollars in 2014 and is predicted to be 42.4 billion dollars in 2019. Factors driving this wire/catheter market includes increasing need for minimally invasive procedures, increasing elderly population, and diseases such as obesity caused by lifestyle which results in the grow of cardiovascular diseases.

Minimally invasive surgery with cardiac catheterization is a main treatment for cardiovascular diseases. Take invasive treatment of coronary heart disease as an example, an arterial sheath (the diameter thereof is about 2 mm to 3 mm) is plugged into an artery in arm or groin, and a steel wire enters a treatment part through the blood vessel to establish a surgery channel. An instrument combined with a plastic catheter enters the blood vessel through the wire for diagnosis and treatment. Therefore, putting the wire into the treatment location through the blood vessel is the first step, and problems in the surgery includes: unidirectional operation which needs many trying; lots of product specification based on the context large amount of X ray and developer are required; depend on doctors experience and high risk. The massive using of X ray and developer may cause other organs (e.g. kidney) damaged. Therefore, it is an issue concerned in the art about how to provide an angiography method which can provide route guide during the surgery with lower amount of X ray and developer.

### SUMMARY

Embodiments of the invention provide a system for angiography. The system comprises an optical fiber, a light emitting module, an ultrasound detector and an imaging system. The optical fiber has a first end and a second end. The second end of the optical fiber is configured to enter a blood vessel of a human body. The light emitting module emits a laser ray into the first end of the optical fiber, and the laser ray diffuses into a human tissue through the optical fiber to generate an ultrasound signal. The ultrasound detector is disposed corresponding to the second end of the optical fiber for receiving the ultrasound signal. The imaging system is coupled to the ultrasound detector and generates an image with respect to the blood vessel according to the ultrasound signal.

In some embodiments, a diameter of the optical fiber is less than or equal to 2 millimeters.

In some embodiments, the system further comprises a wire. A surface of the wire has a linear groove, and the optical fiber is disposed in the linear groove.

In some embodiments, the system further comprises a collar having a first hole and a second hole. The wire penetrate the first hole, and the optical fiber penetrates the second hole.

In some embodiments, the optical fiber has a bonding surface, and the system further comprises a wire and an adhesive. The wire has a bonding surface which is bonded to the bonding surface of the optical fiber. The adhesive is disposed between the bonding surface of the wire and the bonding surface of the optical fiber.

In some embodiments, the optical fiber has bonding surface, and the system comprises a wire having a bonding surface directly connected to the bonding surface of the optical fiber.

In some embodiments, a side surface of the second end of the optical

In some embodiments, a wavelength of the laser ray is ranged between 520 nanometer and 532 nanometer.

From another aspect, an angiography method is provided. The angiography method comprises: emitting, by a light emitting module, a laser ray into a first end of an optical fiber, in which a second end of the optical fiber is configured to enter a blood vessel of a human body, and the laser ray diffuses into a human tissue through the optical fiber to generate an ultrasound signal; receiving, by an ultrasound detector, the ultrasound signal; and generating an image with respect to the blood vessel according to the ultrasound signal.

### BRIEF DESCRIPTION OF THE DRAWINGS

The invention can be more fully understood by reading the following detailed description of the embodiment, with reference made to the accompanying drawings as follows:

FIG. 1 is a schematic diagram illustrating a laser ray emitting out from a blood vessel to perform photoacoustic imaging.

FIG. 2 is a diagram illustrating a partial enlarged view of the second end of the optical fiber according to embodiment.

FIG. 3A to FIG. 3C are diagrams illustrating the bonding between the wire and the optical fiber.

FIG. 4 is a diagram illustrating a flow chart of angiography method according to an embodiment.

### DETAILED DESCRIPTION

Specific embodiments of the present invention are further described in detail below with reference to the accompanying drawings, however, the embodiments described are not intended to limit the present invention and it is not intended for the description of operation to limit the order of implementation. Moreover, any device with equivalent functions that is produced from a structure formed by a recombination of elements shall fall within the scope of the present invention. Additionally, the drawings are only illustrative and are not drawn to actual size.

The using of “first”, “second”, “third”, etc. in the specifications should be understood for identifying units or data described by the same terminology, but are not referred to particular order or sequence.

The present invention is related to an angiography method based on photoacoustic effect. The principle of photoacoustic effect is to irradiate a tissue by light such that the tissue generates a mechanical wave (i.e. ultrasound wave) due to its expansion. The ultrasound wave is received to perform ultrasound imaging. The convention photoacoustic imaging has an issue that light seriously decays with distance so that its imaging distance is short and imaging related researches cannot be performed in a deep position of human body. Therefore, in the angiography method of the invention, an optical fiber is invasively inserted into the blood vessel, and the laser ray is emitted inside the blood vessel such that the human tissue irradiated by the laser ray generates ultrasound waves. As a result, the problem of light decaying with distance is addressed.

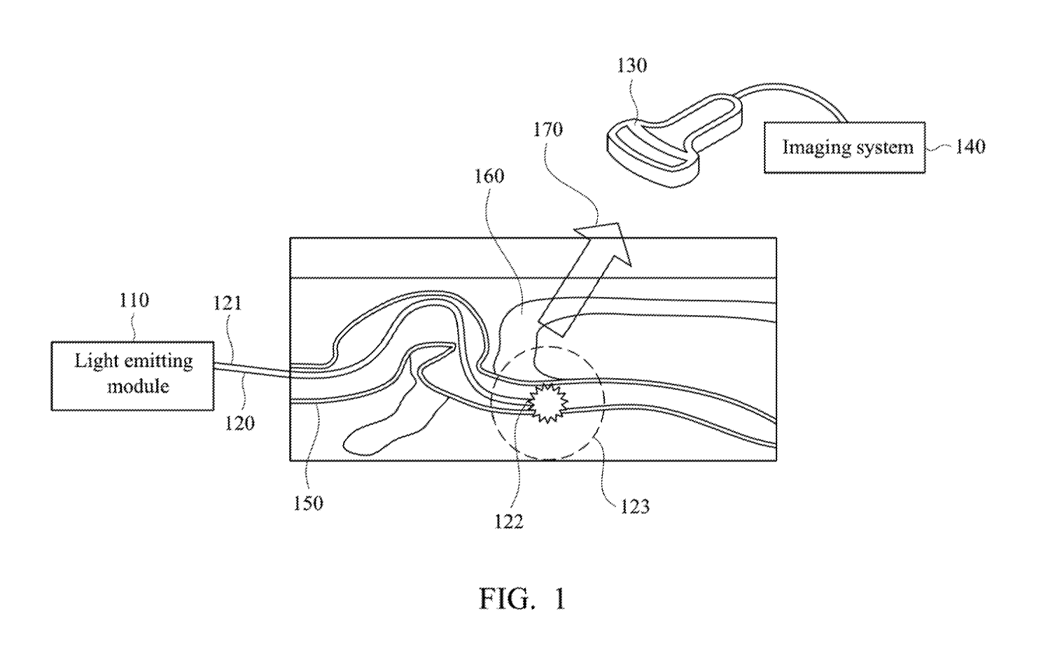

FIG. 1 is a schematic diagram illustrating a laser ray emitting out from a blood vessel to perform photoacoustic imaging. Referring to FIG. 1, the system for angiography includes a light emitting module 110, an optical fiber 120, an ultrasound detector 130 and an imaging system 140.

The optical fiber 120 is configured to enter a blood vessel 150 in a human body. In general, a diameter of the blood vessel 150 is at most 2 millimeters (mm) to 3 mm, and therefore the diameter of the optical fiber 120 has to be less than 2 mm. In the embodiment of FIG. 1, only the optical fiber 120 enters the blood vessel 150, and the optical fiber 120 can bend along with the blood vessel 150. However, in some embodiments, the optical fiber 120 can be bonded to a metal wire so that it would be easier to bend. The embodiment of the wire will be described later.

The light emitting module 110 emits a laser ray into a first end 121 of the optical fiber 120, and thus the laser ray is transmitted through the optical fiber 120, diffuses into a human tissue 160 from a second end 122 of the optical fiber 120. In general, an emitting range 123 of the laser ray in the human body is about 20 mm to 50 mm, and the laser ray would seriously decay out of the emitting range 123. In the embodiment, the laser ray is emitted from the interior of the human body, and therefore the emitting range 123 is enough for the laser ray to irradiate the human tissue 160. Next, the human tissue 160 generates an ultrasound signal 170 due to the photoacoustic effect. It is worth mentioning that the human tissue 160 could be blood vessel, blood or any organs, which is not limited in, the invention.

The ultrasound detector 130 is disposed corresponding to the second end 122 of the optical fiber 120 for receiving the ultrasound signal 170. In general, the propagation range of the ultrasound signal 170 is at least 10 centimeters, and therefore the ultrasound detector 130 can receive clear ultrasound signal 170 even though the ultrasound detector 130 is disposed outside the human body.

The imaging system 140 could be any forms of computer such as personal computer, industry computer and cloud server. The ultrasound detector 130 is coupled to the imaging system 140. The ultrasound detector 130 transforms the ultrasound signal 170 into an electronical signal, and sends the electronical signal to the imaging system 140. A three-dimensional imaging software is performed on the imaging system 140 for illustrating an image with respect to the blood vessel 150 according to the electronical signal transformed from the ultrasound signal 170, and thus the angiography is done. However, which software or algorithm is performed is not limited in the invention. In addition the ultrasound detector 130 is connected to the imaging system 140 through a wire in the embodiment of FIG. 1, but the ultrasound detector 130 may be connected to the imaging system 140 in a wireless way (e.g. through the Internet or any wireless transmission interface).

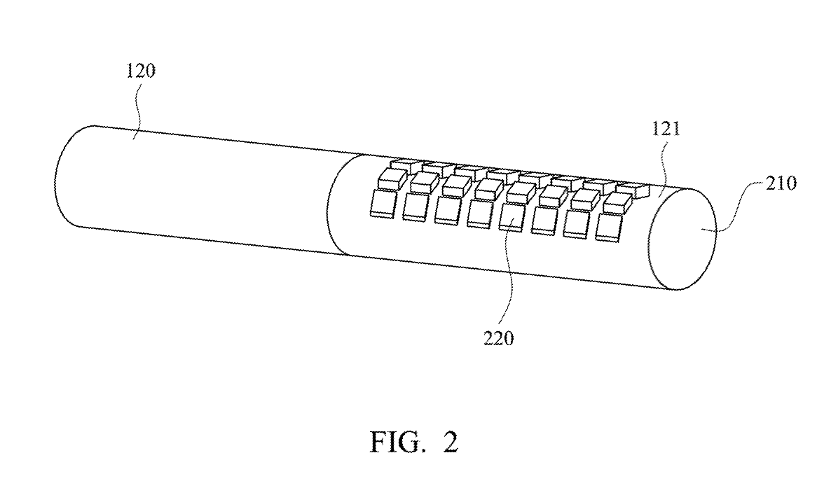

FIG. 2 is a diagram illustrating a partial enlarged view of the second end of the optical fiber according to an embodiment. The laser ray is emitted out only from the top 210 of the second end 121 of the optical fiber 120 in the conventional optical fiber, but the emitting range would be too small. Therefore, some embodiments, a side surface of the second end 121 of the optical fiber 120 has multiple micro-hole structures 220, and the laser ray can emits out from the micro-hole structure 220, resulting in wider emitting range, larger imaging area and better resolution of photoacoustic imaging.

The wavelength of the laser ray may be in any suitable range, and the type of the light emitting module 110 is not limited in the invention. However, different human tissues have different absorption rates for different wavelength of light, and the effect of the photoacoustic imaging is better when the absorption rate is high. For example, blood has relatively higher absorption rate for 520 nm to 532 nm of wavelength, blood vessel has relatively higher absorption rate for about 1064 nm of wavelength, and fat has relatively higher absorption rate for about 1720 nm of wavelength. Although the invention is related to blood vessel imaging, the thickness of blood vessel is generally too thin to generate clear images. Instead, the location of the blood vessel is clearer when the imaging target is blood. Therefore in some embodiment, the wavelength of the laser ray is ranged between 520 nm to 532 m for render blood, and the location of the blood vessel is obtained according to the blood imaging.

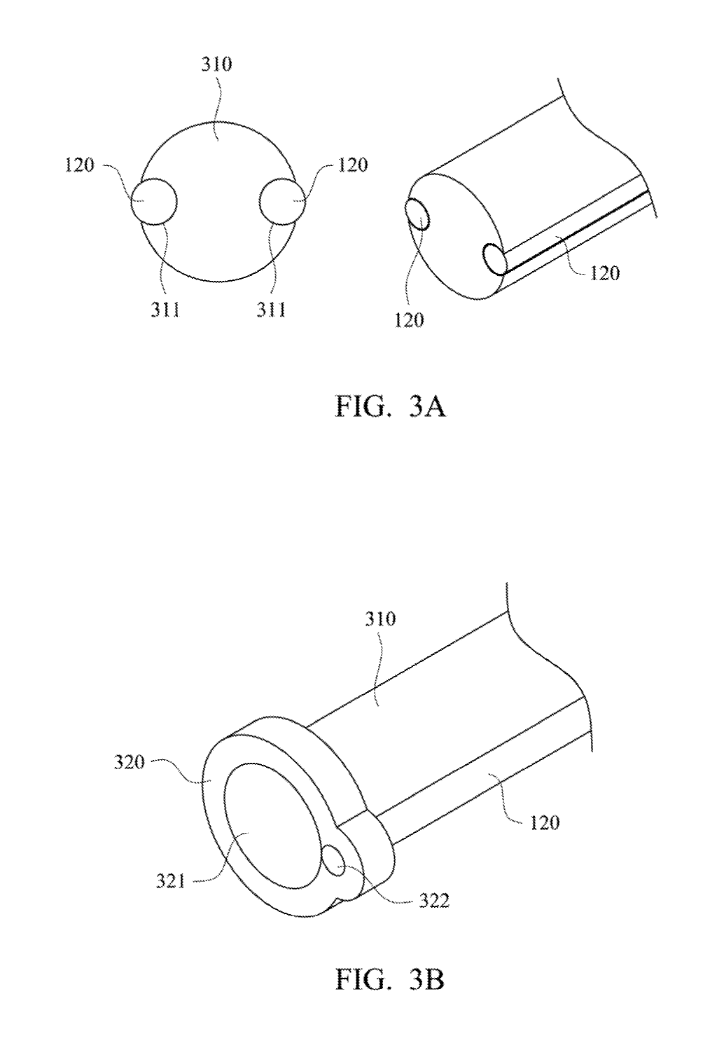

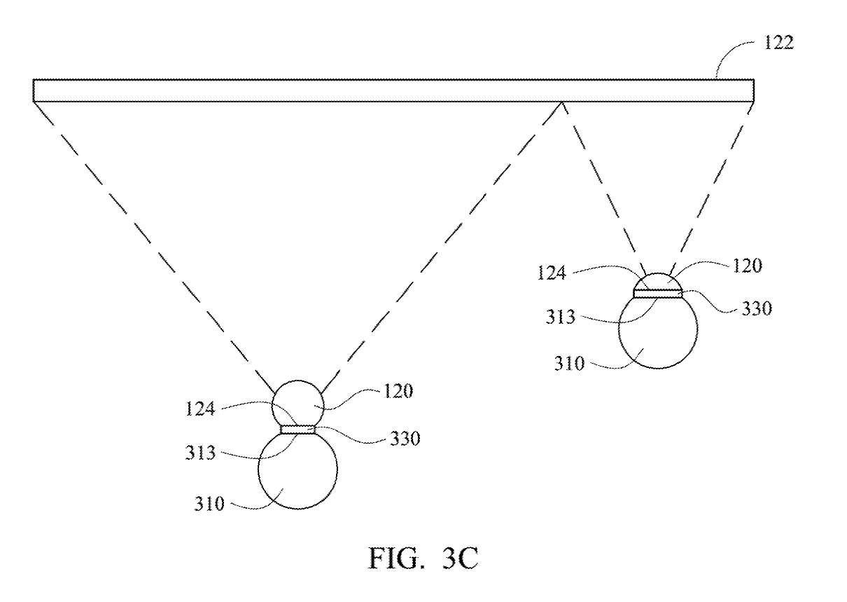

As discussed above, the optical fiber 120 is boned to a wire in some embodiments. Three embodiments are provided bellow to describe the bonding. Referring to FIG. 3A, in the embodiments of FIG. 3A, the surface of the wire 310 has two linear grooves 311, and the optical fibers 120 are embedded into the linear grooves 311. The wire 310 has two linear grooves in this embodiment, but the wire 310 may have more of less linear grooves in other embodiments which is not limited in the invention. It is worth mentioning that because the wire 310 would be inserted into the blood vessel, the diameter of the wire 310 has to be less than 2 mm. For example, the diameter of the wire 310 is 0.35 mm, and the diameter of the optical fiber 120 is 0.125 mm. On the other hand, material of the wire 310 may include any suitable metal, allot, etc., which is not limited in the invention.

In the embodiment of FIG. 3B a collar 320 is used to fix the wire 310 and the optical fiber 120. The collar 320 has a first hole 321 and a second hole 322, the wire 310 penetrates the first hole 321, and the optical fiber 120 penetrates the second hole 322. In some embodiments, more than one collars 320 are disposed on the wire 310 and the optical fiber 120, and these collars are spaced from each other by a fixed or variable distance, and the distance is not limited in the invention. In addition, the largest diameter of the collar 320 is less than 2 mm.

In the embodiment of FIG. 3C, the wire 310 has a bonding surface 313, the optical fiber 120 has a bonding surface 124, and the bonding surface 313 is bonded to the bonding surface 124. In addition, an adhesive 330 is disposed between the bonding surface 313 and the bonding surface 124 to fix the wire 310 and the optical fiber 120. The adhesive 330 may be biological adhesive or other bonding material capable of being disposed in the human body. From another aspect, the bonding surface 313 seems as a chord of a circle when viewed from a cross-section of the wire 310, and the chord length is variable. In the embodiment of FIG. 3C, the chord length corresponding to the bonding surface 313 near the second end 122 is relatively larger, and the chord length corresponding to the bonding surface 313 far from the second end 122 is relatively smaller because longer chord makes the diameters of the wire 310 and the optical fiber 120 smaller. The second end 122 is configured to enter the human body, and it needs the smaller diameter.

In another embodiment, the wire 310 has the bonding surface 313, the optical fiber 120 has the bonding surface 124, but no adhesive 330 is disposed. Instead, the bonding surface 313 of the wire 310 is directly connected to the bonding surface 124 of the optical fiber 120. For example, the bonding surface 313 of the wire 310 is directly connected to the bonding surface 124 of the optical fiber 120 by welding, but the invention is not limited thereto.

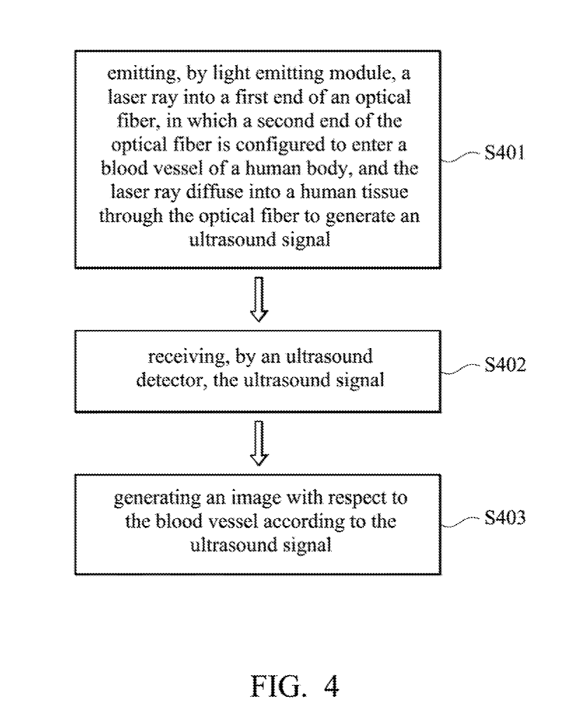

FIG. 4 is a diagram illustrating a flow chart of angiography method according to an embodiment. The steps could be implemented as programs and executed by the imaging system 140 which can control the light emitting module 110. In step S401, emitting, by a light emitting module, a laser ray into a first end of an optical fiber, in which a second end of the optical fiber is configured to enter a blood vessel of a human body, and the laser ray diffuse, into a human tissue through the optical fiber to generate an ultrasound signal. In step S402, receiving, by an ultrasound detector, the ultrasound signal. In step S403, generating an image with respect to the blood vessel according to the ultrasound signal. However, each step of FIG. 4 has been described in detail above, and therefore the description will not be repeated.

Although the present invention has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present invention without departing from the scope or spirit of the invention. In view of the foregoing, it is intended that the present invention cover modifications and variations of this invention provided they fail within the scope of the following claims.

What is claimed is:

1. A system for angiography, comprising:
an optical fiber, having a first end and a second end, wherein the second end of the optical fiber is configured to enter a blood vessel of a human body;
a light emitting module, configured to emit a laser ray into the first end of the optical fiber, wherein the laser ray diffuses, into a human tissue through the optical fiber to generate an ultrasound signal;
an ultrasound detector, disposed corresponding to the second end of the optical fiber for receiving the ultrasound signal; and
an imaging system, coupled to the ultrasound detector and generating an image with respect to the blood vessel according to the ultrasound signal.

2. The system of claim 1, wherein a diameter of the optical fiber is less than or equal to 2 millimeters.

3. The system of claim 1, further comprising:
a wire, wherein a surface of the wire has a linear groove, and the optical fiber is disposed in the linear groove.

4. The system of claim further comprising:
a wire; and
a collar, having a first hole and a second hole, wherein the wire penetrates the first hole, and the optical fiber penetrates the second hole.

5. The system of claim 1, wherein the optical fiber has a bonding surface, and the system further comprises:
a wire, having a bonding surface, wherein the bonding surface of the wire is bonded to the bonding surface of the optical fiber; and
an adhesive, disposed between the bonding surface of the wire and the bonding surface of the optical fiber.

6. The system of claim 1, wherein the optical fiber has a bonding surface, and the system comprises:
a wire, having a bonding surface directly connected to the bonding surface of the optical fiber.

7. The system of claim 1, wherein a side surface of the second end of the optical fiber has a plurality of micro-hole structures.

8. The system of claim 1, wherein a wavelength of the laser ray is ranged between 520 nanometer and 532 nanometer.

9. An angiography method for an imaging system, the angiography method comprising:
emitting, by a light emitting module, a laser ray into a first end of an optical fiber,wherein a second end of the optical fiber is configured to enter a blood vessel of a human body, and the laser ray diffuses into a human tissue through the optical fiber to generate an ultrasound signal;
receiving, by an ultrasound detector, the ultrasound signal; and
generating an image with respect to the blood vessel according to the ultrasound signal.
